(19) United States
(12) Patent Application Publication
(10) Pub. No.: US 2016/0106920 A1
(43) Pub. Date: Apr. 21, 2016

(54) Medical Injection Device

(71) Applicant: Novo Nordisk A/S, Bagsvaerd (DK)

(72) Inventor: Mads Schenstroem Stefansen, Copenhagen OE (DK)

(21) Appl. No.: 14/895,155

(22) PCT Filed: May 27, 2014
(86) PCT No.: PCT/EP2014/060924

§ 371 Date: Dec. 1, 2015

Related U.S. Application Data
(60) Provisional application No. 61/833,070, filed on Jun. 10, 2013.

(30) Foreign Application Priority Data
Jun. 3, 2013 (EP) 13170328.2

(51) Int. Cl. A61M 5/20; A61M 5/32

(52) U.S. Cl. CPC A61M 5/2033; A61M 5/3287; A61M 5/3245; A61M 5/326; A61M 2005/2013; A61M 2005/2418

(57) ABSTRACT

The present invention relates to a medicament injection device (100) that comprises a housing (110, 120) and an actuator (400, 500) configured for expelling medicament from a held reservoir (200) through an injection needle (250). A needle shield (150) is movable from a distal position through an intermediary position and further proximally to activate dose expelling. A carrier (300) movable within the housing (110, 120) holds the injection needle (250). A detent mechanism couples carrier (300) with the needle shield (150) as the needle shield (150) moves from the distal to the intermediary position. When the needle shield is moved further proximally the detent mechanism decouples the carrier (300) from the needle shield (150). A carrier spring device (680) is arranged between the housing (110, 120) and the carrier (300). The injection device (100) provides increased safety against potential damage to the injection needle (150) in case the injection device (100) is dropped on a hard surface.

The present invention relates to injection devices for injecting one or more dose of a liquid medicament. In particular the present invention relates to injection devices for injecting a medicament from a held medicament reservoir by means of an injection needle and improvements relating to the safety of the injection device during handling and transportation.

### BACKGROUND OF THE INVENTION

In relation to some diseases patients must inject a medicament on a regular basis such as once weekly, once daily or even a plurality of times each day. In order to help patients overcome fear of needles, fully automatic injection devices have been developed that makes the use of an injection device as simple as possible. Auto-injectors of this kind are typically designed so that a user only needs to position the injection device onto the injection site and activate the device. Such activation causes the device to insert a needle into the skin and expel a dose of the medicament. Optionally, subsequently to the expelling of the dose, the needle may be brought into a shielded state.

Some auto-injectors provide auto-insertion of the needle into the skin of the user, e.g. by means of a spring actuated insertion mechanism. However, auto-injectors that provide automatic insertion of the needle into the dermis also prevent the user from controlling the insertion, which can lead to uneasiness for the user. Other auto-injectors, such as the injection devices disclosed in WO 2012/022810, provide manual needle insertion where the user of the device is offered full control of the insertion process. Typically, the needle insertion is provided by means of a needle shield that initially protects the front part of the needle. Movement of the needle shield relative to the housing of the device gradually exposes the front part of the needle. During this relative movement the needle is introduced into the skin.

Some auto-injectors use a dedicated release button for triggering the expelling process. Contrary to this front triggered injection devices combine the needle insertion with the actuation of the injection. From the users point of view the device consist of a main body and a needle shield. The user only have to place and point on the injection spot with the needle shield and press the main body towards the skin. This makes especially—but not only—single dose devices very simple and easy to use. Front triggered devices have the disadvantage that they might be accidentally triggered when dropped. An easy solution would be to add an additional release button. However; this will significantly increase the complexity of the use of the device which might exclude some users with dexterity problems. Furthermore there is always a risk of damaging the needle if the device is dropped.

Having regard to the above-identified prior art devices, it is an object of the present invention to provide an injection device which enables simplified handling, improved control of the device during handling thereof and yet offers improvements relating to the safety of the device.

Yet additional further objects of the invention are to provide measures for obtaining devices having a superior performance and, at the same time, enabling manufacture at a reduced cost.

### BRIEF DESCRIPTION OF THE INVENTION

In a first aspect, the present invention relates to an injection device for administering a dose of a medicament from a held medicament reservoir, the injection device comprising:

a) a housing defining distal and proximal ends,

b) an injection needle arranged at the distal end of the housing and adapted for fluid communication with a held medicament reservoir,

c) a needle shield disposed at the distal end of the housing, the needle shield being movable proximally relative to the housing upon exertion of an externally applied force to gradually expose the injection needle in the course of the needle shield being moved proximally relative to the housing, and

d) an actuator configured to act on the medicament reservoir to expel a dose of medicament therefrom.

The injection device further comprises:

e) a carrier that is movable relative to the housing from a distal position to a proximal position, the carrier holding the injection needle,

f) a carrier spring device arranged between the housing and the carrier, the carrier spring device being configured to force the carrier in the distal direction, and

g) a detent mechanism arranged between the carrier and the needle shield. The the detent mechanism is configured to: I) couple axial movement of the needle shield with axial movement of the carrier as the needle shield moves proximally relative to the housing from a distal position to an intermediary position so that the injection needle is maintained in a shielded state, and II) allow relative axial movement between the needle shield and the carrier as the needle shield moves relative to the housing from the intermediary position and into a proximal position so that the injection needle is gradually exposed as the needle shield moves relative to housing between the intermediary position and the proximal position.

The needle shield may define a distal end face that is adapted to rest against the skin at an injection site. In the context of the present disclosure, when referring to the needle being gradually exposed, it should be so construed that a gradually larger portion of the needle extends distally relative to the distal end face of the needle shield as the needle shield moves proximally relative to the housing. The gradually larger exposed portion of the needle allows said exposed portion of the needle to protrude into an injection site.

In a situation of use, said externally applied force is exerted by the hand of a user when the user forces the housing of the device in the distal direction towards the surface of an injection site.

As the needle shield moves from the distal position to the intermediary position relative to the housing the travel of the needle shield is accompanied by movement of the carrier, for example so that the needle shield and the carrier moves in unison. During this travel the carrier spring device takes up energy as it is being increasingly tensed. When the needle shield is moved from the distal position to the intermediary position, the carrier, the injection needle, and optionally also the medicament reservoir and the actuator all move with the needle shield relative the housing. Thus the weight of components which is rigidly connected to the housing is comparatively low. Hence, in case the injection device is dropped on a hard surface resulting in a proximally directed impact on the needle shield the risk of such impact resulting in the device being unintentionally activated for expelling of a dose is low.

With such an injection device it is possible to make a device that is front triggered and have manual needle insertion but have the safety benefits of a device with an activation button, thus keeping the number of use steps to a minimum. At the same time it also protects the injection needle against damages caused by an unintentional impact.

In some embodiments the housing of the injection device forms a substantial part of the outer surface of the device and is adapted to be gripped by the hand of the user. Hence, the injection device is operated by gripping the device with a hand and applying the needle shield onto an injection site, i.e. into skin contact. The injection device is then forced towards the skin resulting firstly in the manual insertion of the needle into the skin and subsequently the activation of the actuator which carries out the expelling operation.

In some forms the detent mechanism is configured to prevent relative axial movement between the carrier and the needle shield when the needle shield is positioned between the distal position and the intermediary position.

The detent mechanism may be coupled to the needle shield and the carrier to lock the carrier relative to the needle shield as long as the needle shield is moved from the distal position and towards the intermediary position. A carrier control member may be arranged to cooperate with the housing so that relative movement between the needle shield and the housing acts to release said lock when the needle shield, upon proximal movement, enters into the intermediary position.

The needle shield may define a trigger position relative to the housing where the trigger position is located proximally to said intermediary position. The actuator is maintained in an inactive state when the needle shield is positioned distally relative to the trigger position. When the needle shield is moved relative to the housing into its trigger position, the actuator is released from the inactive state, enabling expelling of a dose of the medicament.

The actuator may include a piston driver adapted to act on the medicament reservoir to expel a dose of medicament therefrom. The actuator may also include an energy source which upon release drives the piston driver to expel medicament. The energy source may be provided as a stored energy source, such as a pre-strained spring, a compressed gas etc. In other forms, the energy source is configured to become charged during an initial operation of the device prior to activation of the injection mechanism. In still other embodiments, the actuator may be provided as a device which is manually driveable by the user of the device, e.g. by coupling the manually driveable device with the piston driver or by providing the piston driver as the manually driveable device.

In some configurations, the carrier spring may be provided separately from the energy source of the actuator and may be configured so that the carrier spring is operated separately from the energy source, when the carrier is moved in proximal direction relative to the housing.

In embodiments where the actuator comprises a stored energy source which is so configured that, upon release, energy from the stored energy source drives the piston driver to expel medicament, the needle shield may be so configured that, upon the needle shield being moved proximally relative to the housing into its trigger position, the actuator is automatically released from the inactive state causing expelling of a dose of the medicament.

Having regard to the actuator of the device, the energy source of the actuator may comprise a spring that is adapted to drive the piston driver in the distal direction. The spring may be a spring device that either works in a compression mode, in a torsion mode or in a combined compression and torsion mode. The spring may be a pre-tensed spring which is tensed during manufacture of the device. Alternatively, the device may include a mechanism for tensioning the spring as an initial procedure when taking the device into use. Alternatively, the energy source of the actuator may be in the form of a compressed medium such as a gas. Still alternatively, the actuator may include a gas generator such as an electro-chemical cell.

The needle shield may be so configured that movement of the needle shield acts to release the actuator during II) in a state where relative movement between the needle shield and the carrier has shifted the injection needle into the state where the injection needle is at least partly exposed. In some embodiments, the release occurs when a substantive part of the injection needle protrudes from the needle shield, such as by 3, 4, 5, 6, 7, 8, 9, 10, 11, 12, 13, 14 or 15 mm from a distal end face of the needle shield.

The needle shield may be coupled to a needle spring device configured to bias the needle shield towards its distal position relative to the housing. The needle spring device may be arranged between the needle shield and the housing. In other embodiments needle spring may be situated at other locations. Also, the spring action provided by needle spring may be obtained by other means such as an elastic foam member, a pneumatic spring, a magnetic force or the like.

In some embodiments an impact damper is arranged between the housing and the carrier, where the impact damper is configured to dampen motion of the carrier relative to the housing.

The impact damper may provide a damping effect on axial movements of the needle shield with respect to housing. In accordance herewith, the impact damper provides a damping effect on the needle shield so that sudden impacts which potentially may be exerted on needle shield, such as when the injection device is unintentionally dropped on a hard surface, reduces the risk of the injection device being prematurely triggered.

Should an unintentional impact on needle shield occur, then the impact damper will be able to limit part of the impact forces to be transferred into movement of the needle shield and thus limit the extent of movement of the needle shield. However, when the injection device is applied to an injection site such that the needle shield is slowly pressed in the proximal direction relative to housing, the impact damper allows the needle shield to be moved from the distal position to the proximal position with little or no damping effect. Hence, when the needle shield is pressed slowly in the proximal direction, the needle shield is allowed to progress as far as to the intermediary position relative to the housing. Hereafter the needle shield can move further in the proximal direction for bringing the device into the triggered state. This latter movement is unrestricted by the impact damper and the carrier spring device due to the needle shield has become disengaged relative to the carrier.

The impact damper may be configured as a damper mechanism incorporating one or more operating principles selected from the group consisting of a hydraulic damper, a pneumatic damper, a grease damper, a rotational motion damper and a foam damper.

In some embodiments, the impact damper comprises one part that is associated or coupled directly with the carrier while a second part is associated or coupled directly to the housing. In other embodiments the impact damper is coupled to the carrier by means of the needle shield. Such embodiment may include an impact damper coupled between the housing and the needle shield to operate on the carrier while the needle shield is positioned between the distal position and the intermediary position. In still other embodiments, an impact damper is coupled between the housing and the needle shield to act on the needle shield while the needle shield is positioned between the distal position and the intermediary position and wherein the impact damper releases from the needle shield upon the needle shield moving further proximally to render the needle shield operate without the damping effect of the impact damper.

In some embodiments the medicament reservoir defines an elongated reservoir body where the injection needle is mounted on said reservoir body at a distal end thereof and wherein a slideable piston is arranged within the reservoir body to be driveable in the distal direction to expel medicament from the medicament reservoir.

In particular embodiments, the needle is fixedly attached to the carrier. The carrier may further be adapted to hold the medicament reservoir. The medicament reservoir may, in a situation of use, be fixedly mounted relative to the carrier. The body of the medicament reservoir may be formed by glass or a synthetic resin. The reservoir may be of the type having a needle fixedly attached to the body of the medicament reservoir.

In other embodiments the medicament reservoir defines a medicament cartridge having an elongated cartridge body, wherein a cartridge septum seals the distal end of cartridge body, and wherein the cartridge septum is configured for being pierced by the injection needle for establishing fluid communication with medicament contained in the cartridge. A slideable piston is arranged within the cartridge body to be driveable in the distal direction to expel medicament from the medicament cartridge.

The injection needle may define a front needle for penetrating the skin of a subject user and a rear needle for piercing the cartridge septum. The front needle and rear needle is configured for fluid communication. Such injection needle may be mounted fixedly relative to the carrier so that the injection needle is moveable together with the carrier.

The medicament cartridge may be arranged relative to or within said carrier so that, prior to triggering of the injection device, i.e. prior to operation of the actuator, the rear needle is separated axially from the cartridge septum. The medicament cartridge may be arranged slideable within the carrier so that when the actuator acts on the medicament cartridge, the medicament cartridge initially slides relative to the carrier so that the cartridge septum is pierced by the rear needle and subsequently the piston is driven by the actuator in the distal direction relative to the cartridge body to expel medicament from the medicament cartridge. In such embodiments, before operation of the actuator, the medicament cartridge may be arranged to follow movements of the carrier as the carrier is moved relative to the housing.

In some embodiments, the injection device forms a device wherein a single dose of injection may be administered whereafter the device is discarded. The needle shield may be so configured that, subsequent to an injection and as the injection device is withdrawn from the injection site, the needle shield automatically moves distally and permanently locks in a position where the front end of the injection needle is inaccessible by the hands of the user.

In other embodiments the injection device may be configured for multiple separate injections whereby the needle shield is adapted multiple times to be moved back and forth relative to the housing from a distal position to a proximal position and where the carrier moves with the needle shield from the distal position to the intermediary position and where the needle shield is decoupled from the carrier to allow the needle shield to move further proximally independent from the needle carrier. During the latter movement the injection needle is brought to protrude from the needle shield whereafter the injection device is triggered for dose expelling by moving the needle shield even further proximally.

In still other embodiments according to the invention, the size of the dose of the single dose, or, in case the injection device is configured for multiple injections, the size of individual doses may be adjustable by the user by means of a dose setting feature whereby the user may adjust the size of a dose that subsequently is expelled.

### DETAILED DESCRIPTION OF THE INVENTION

The invention will now be described in further detail with reference to the drawings in which:

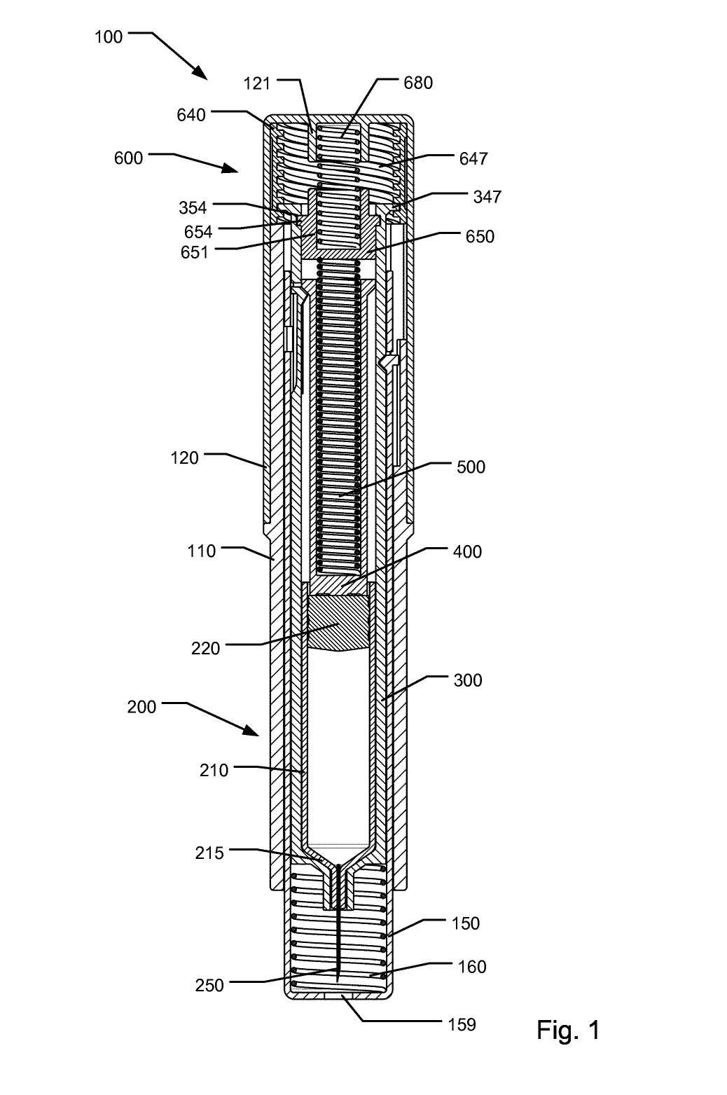

FIG. 1 shows a side sectional view of an injection device according to a first embodiment of the invention,

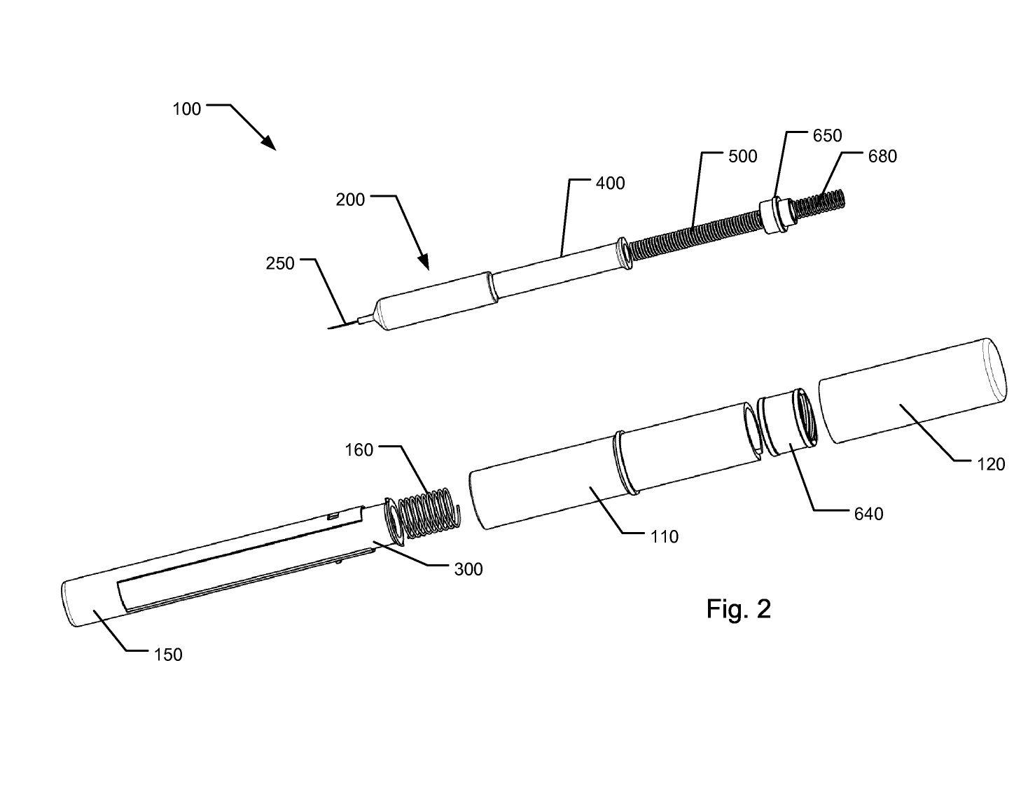

FIG. 2 shows an exploded perspective view of the injection device according to the first embodiment,

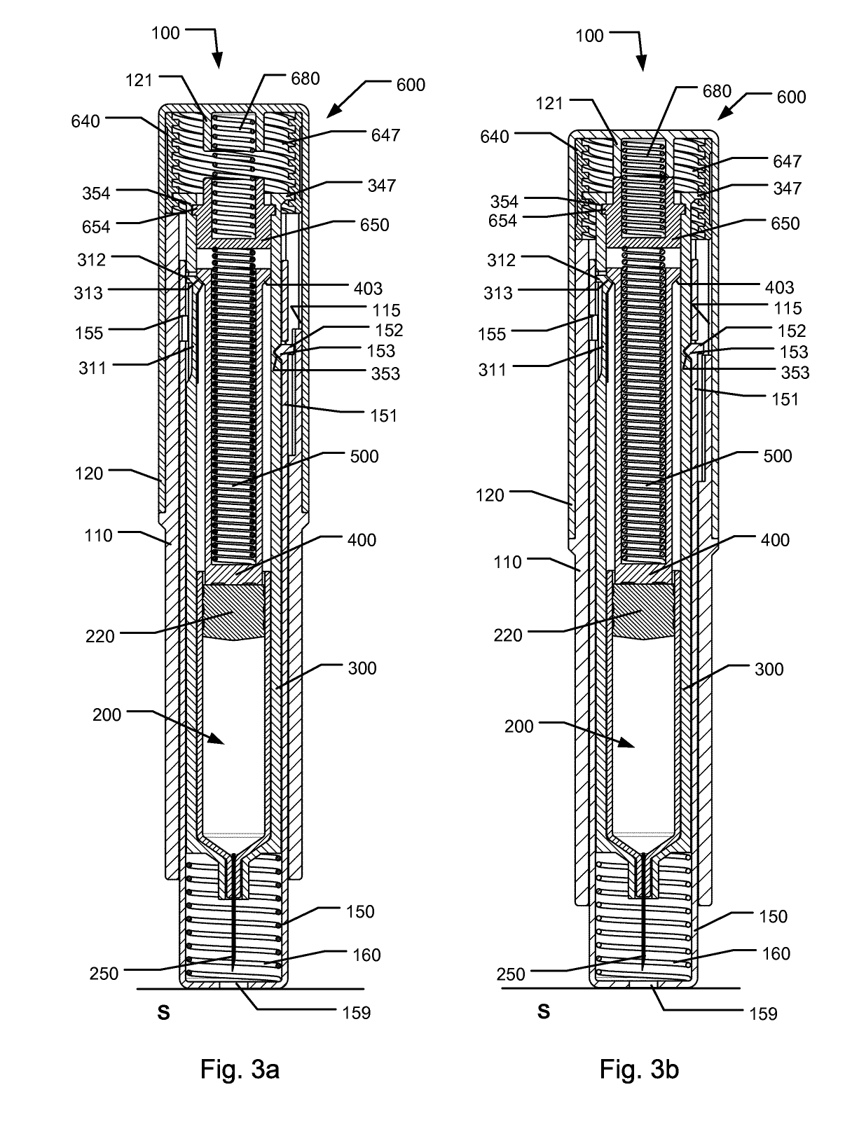

FIGS. 3a through 3e depict side sectional views of an injection device according to the first embodiment of the invention, each view showing the device in a respective operational state,

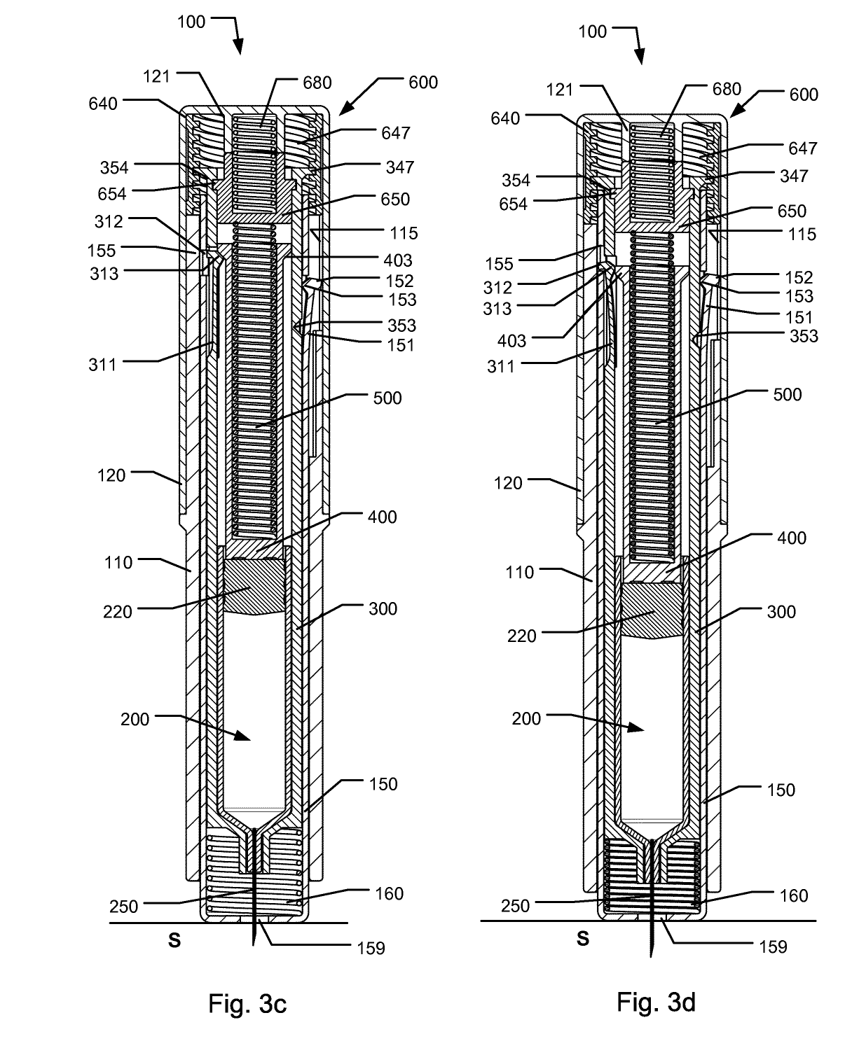

FIG. 4 shows a side sectional view of an injection device according to a second embodiment of the invention,

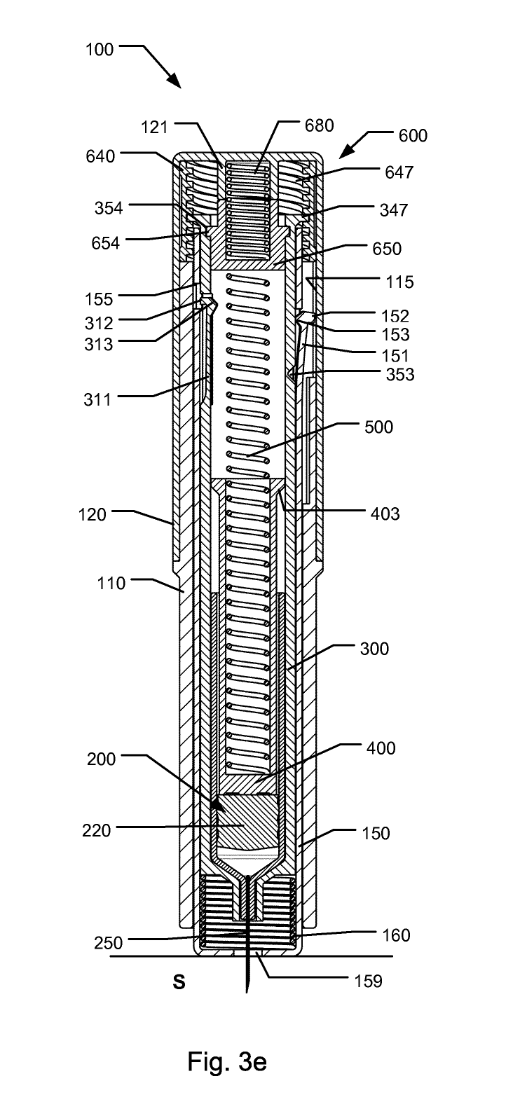

FIG. 5 shows a side sectional view of an injection device according to a third embodiment of the invention,

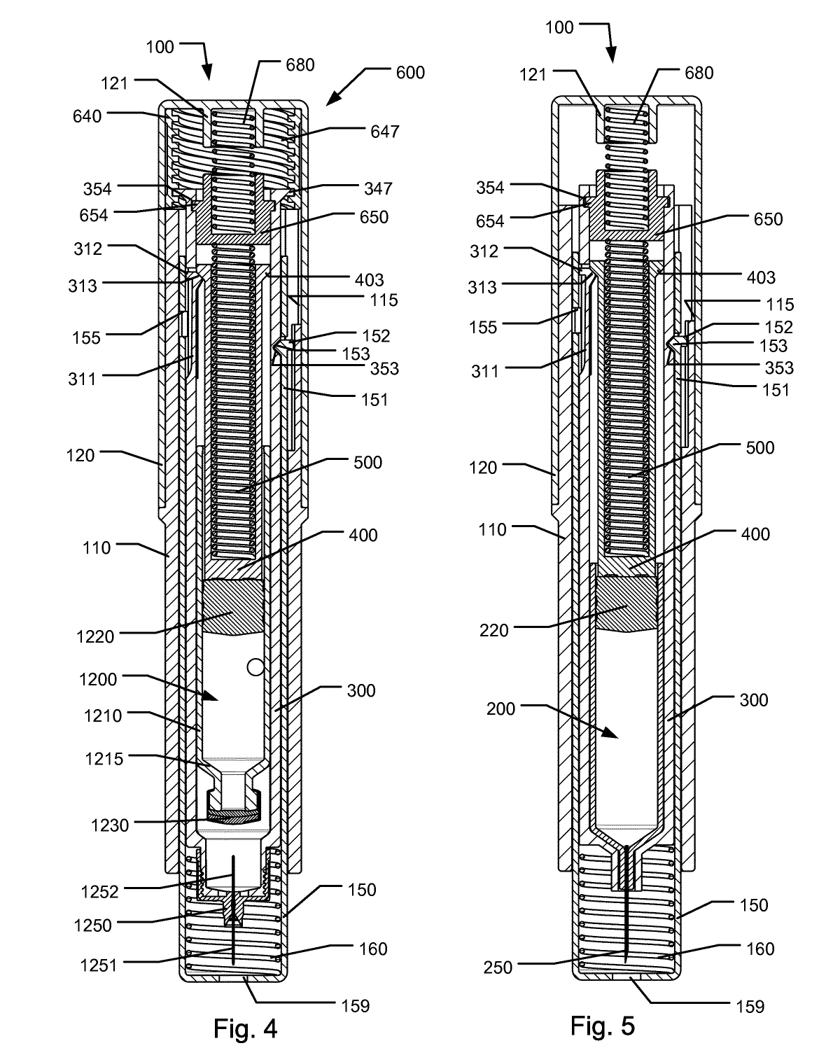

FIG. 6 shows a side sectional view of an injection device according to a fourth embodiment of the invention,

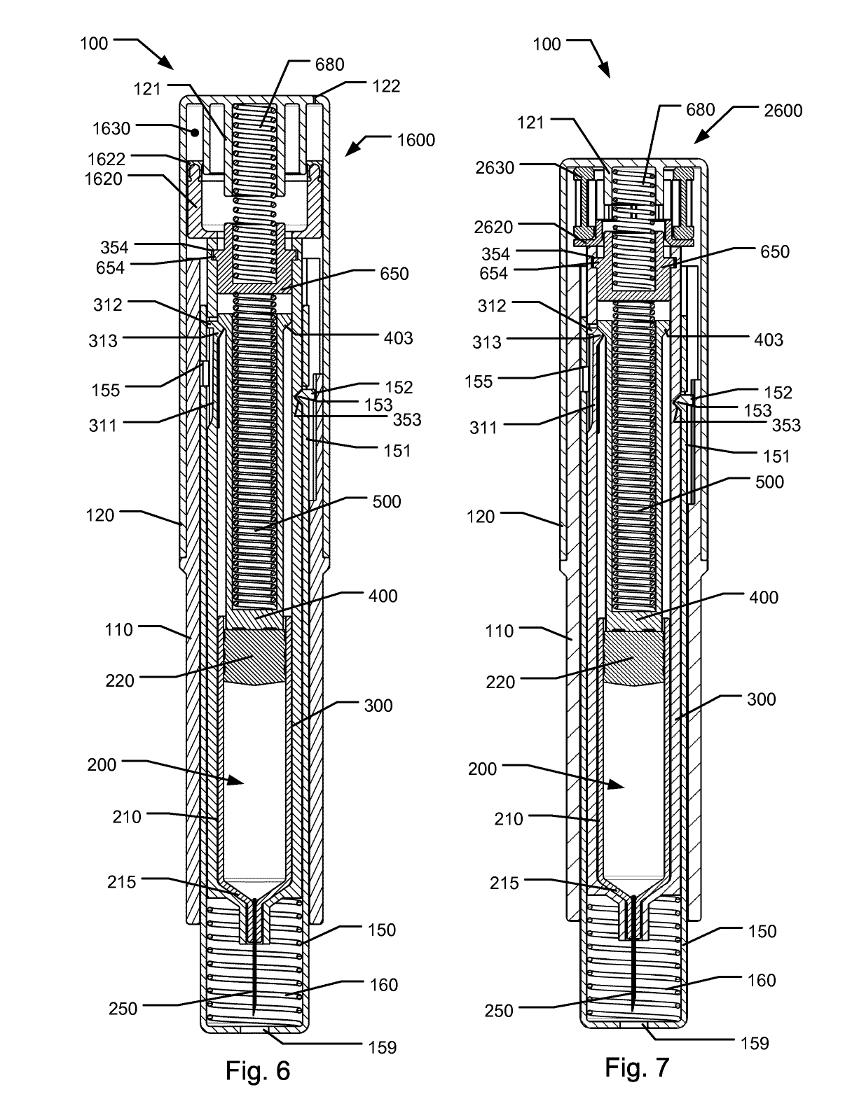

FIG. 7 shows a side sectional view of an injection device according to a fifth embodiment of the invention, and

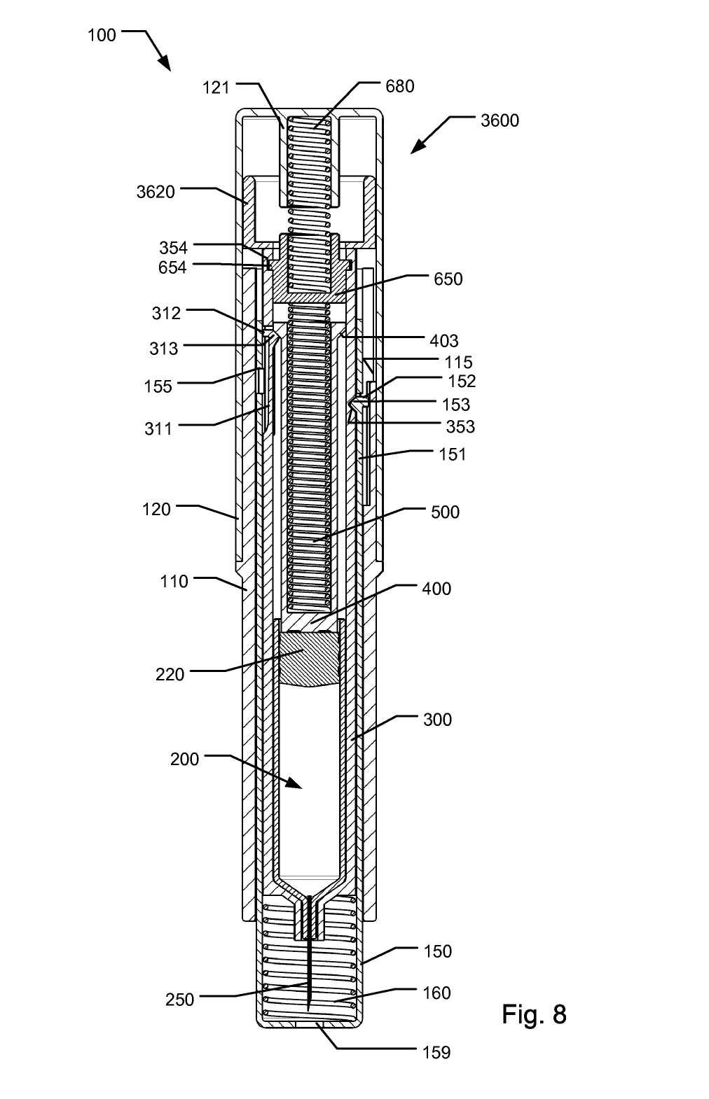

FIG. 8 shows a side sectional view of an injection device according to a sixth embodiment of the invention.

In the context of the present disclosure it may be convenient to define that the term “distal end” in the appended figures is meant to refer to the end of the injection device which usually carries the injection needle whereas the term “proximal end” is meant to refer to the opposite end of the injection device pointing away from the injection needle. The shown figures are schematical representations for which reason the configuration of the different structures as well as the relative dimensions are intended to serve illustrative purposes only.

FIG. 1 shows a first embodiment of a medical injection device 100 for injecting a pre-determined amount of a liquid medicament from a held medicament reservoir 200 through an injection needle 250. The injection device 100 includes a dose expelling mechanism for expelling a dose from the medicament reservoir. The depicted embodiment shown in FIG. 1 is provided as an auto-injector where energy is stored in a pre-stressed spring, where a needle shield is operated to activate the device to thereby trigger the expelling procedure for injecting a fixed dose of medicament from the medicament reservoir.

FIG. 2 shows an exploded perspective view of the main components of the injection device 100.

Referring to FIGS. 1 and 2, the injection device 100 includes an elongated generally tubular housing comprising a main housing 110 and a top housing 120 arranged at a proximal end of main housing 110, the tubular housing defining a central longitudinal axis. The housing 110/120 accommodates a medicament reservoir 200 in form of a prefilled syringe having an injection needle 250 integrally mounted therewith. Syringe 200 includes a cylindrical body 210 that includes a narrowed neck section 215 at its distal end where the injection needle 250 is mounted. Near its proximal end, body 210 accommodates an elastomeric piston 220 that seals the syringe chamber formed by cylindrical body 210. Piston 220 may be pushed in the distal direction to expel liquid from the syringe chamber. A removable protective cap (not shown) may be arranged at a distal end of injection device 100 for protecting the needle end of the device 100 and, optionally during storage of the device, for providing a sealing function for the injection needle 250. The main housing 110 includes two opposing windows (not shown) which allow visual inspection of the medicament contained within medicament reservoir 200. In addition, the said windows enable a user of the device to determine whether or not the device 100 has already been used for a dose expelling operation. Due to manufacturing reasons, in the shown embodiment, top housing 120 is formed as an element separate from but permanently fixed to main housing 110. In alternative embodiments top housing 120 is formed integral with main housing 110.

FIG. 1 depicts the device 100 in the initial state, i.e. after the protective cap has been removed but prior to medicament administration. Shown protruding from the distal end of the main housing 110 is a needle shield 150 that is arranged coaxially relative to main housing and slideable relative to the housing between a distal (extended) position and a proximal (compressed) position. When the needle shield 150 is in the distal position, the injection needle 250 is in a shielded state whereas when the needle shield is in the proximal position the front end of the injection needle 250 protrudes through an aperture 159 arranged in the central part of a distal face of the needle shield 150 so that at least part of the front end of the injection needle 250 extends distally from the distal face of the needle shield and is thus exposed. In the shown embodiment, as will be discussed later, the needle shield 150 additionally serves as an activator for triggering the dose expelling operation.

The protective cap, when attached to the main housing 110, prevents the needle shield 150 from being manipulated and thereby prevents triggering of the injection device 100. In the shown embodiment, when mounted onto the device, the protective cap attaches relative to not shown fastening geometries of the main housing 110 but the protective cap does not cooperate with the needle shield 150. In other embodiments, the protective cap, when mounted on the device 100, cooperates and engages with the needle shield to maintain the position of the needle shield relative to the housing of the device. A non-limiting example of such retaining function may be provided by forming the protective cap with a thread that engages a thread formed on the needle shield and wherein an additional thread of the protective cap engages a thread associated with the housing of the device. By making the two threaded engagements of similar lead the needle shield will be retained axially relative to the main housing when the protective cap is in the mounted position, whereas when the protective cap is removed by turning the protective cap relative to the housing of the device, the needle shield is allowed to move axially relative to the main housing.

A carrier 300 is situated within housing 110/120 in manner that enables carrier 300 to be shifted back and forth relative to the housing along the central axis. In the shown embodiment, carrier 300 forms an elongated cylindrical enclosure that accommodates medicament reservoir 200 so that the medicament reservoir is positioned distally with the injection needle 250 protruding distally relative to carrier 300. A carrier end member 650 is fixedly mounted relative to carrier 300 at the proximal end thereof. In the shown embodiment, carrier end member 650 defines a radially outwards circular rim 654 which fits into a correspondingly shaped internal recess 354 formed in a radially inward facing surface of the carrier 300. In this way the carrier end member 300 may be mounted by means of a snap connection to provide an axial fixation of carrier end member 650 relative to carrier 300. In the shown embodiment carrier 300 is mounted relative to the housing 110/120 so as to prevent rotational movement. Also needle shield 150 is mounted relative to the housing 110/120 so as to prevent relative rotational movement.

Inside carrier 300, proximally to the piston of the medicament reservoir 200 and coaxially therewith, a piston driver 400 is arranged. Piston driver 400 is formed as an elongated member having a length suitable for driving piston 220 from its initial position to its desired end position. Piston driver 400 defines a proximally facing cylindrical bore. A first compression spring 500 is arranged between the carrier end member 650 and the piston driver 400 so that, when the injection device 100 is in its initial state, a substantial part of the first compression spring 500 is accommodated in the bore of the piston driver 400. When the injection device 100 is in the initial state, i.e. prior to triggering of the device, the compression spring 500 is maintained in a pre-stressed condition exerting a distally directed force on piston driver 400. However, in the initial state, the piston driver 400 is retained relative to the carrier 300 but will upon triggering of the device exert a distally directed force on piston 220.

The carrier end member 650 forms at its proximally facing end surface a cylindrical bore defining a first spring support 651. Likewise, the top housing 120 at its proximal end includes a distally facing end surface which forms a cylindrical bore defining a second spring support 121. A carrier spring device provided in the form of a second compression spring 680 is arranged between the first spring support 651 and the second spring support 121, each of the ends of the second compression spring 680 fitting into the respective cylindrical bore of the first and second spring support. In this way the second compression spring 680 is adapted to provide a distally directed force on the carrier 300 to urge the carrier towards the distal end of housing 110/120. As will later be described, the injection device 100 includes means for ensuring that carrier 300 will only be axially moveable between a proximal end position and a distal end position relative to housing 110/120.

Between the needle shield 150 and carrier 300 a needle shield spring 160 is arranged to exert a distally directed force on needle shield 150 so as to urge the needle shield towards its distal position. In the shown embodiment, the needle shield spring 160 is arranged distally of carrier 300. However, in other embodiments needle spring may be situated at other locations. Also, the spring action provided by needle spring 150 may be obtained by other means such as an elastic foam member, a pneumatic or hydraulic spring device, a device utilizing magnetic force or the like.

In the shown embodiment, when the needle shield 150 is pushed in the proximal direction relative to housing 110/120, such as when the injection device 100 with the extended needle shield 150 is pressed towards an injection site, carrier 300 will initially be forced proximally against the force provided by the second compression spring 680. Hence, potential impact forces acting to urge the needle shield in the proximal direction will be counteracted by the second compression spring 680.

In the embodiment shown in FIG. 1, an impact damper generally referenced 600 has been included in device 100 to provide a damping effect on axial movements of the needle shield 150 with respect to housing 110/120. In accordance herewith, the impact damper 600 provides a damping effect on the needle shield 150 so that sudden impacts which potentially may be exerted on needle shield 150, such as when the injection device 100 is unintentionally dropped on a hard surface, reduces the risk of the injection device 100 being prematurely triggered. In the device shown in FIG. 1 the impact damper is provided as a rotational motion damper that transfers axial motion energy into rotational motion energy.

Rotatable member 640 is positioned in proximal hollow of top housing 120 and arranged axially fixed relative to housing 110/120 but able to rotate around the central axis of device 100. Rotational member 640 includes one or more internal thread segments 647 providing a threaded engagement with corresponding one or more thread segments 347 arranged to protrude radially outwards on a proximal portion of carrier 300. The threaded engagement is provided as a not self-locking thread whereby relative axial movement between carrier 300 and housing 110/120 will induce a rotational movement of rotatable member 640. This design performs as an impact damper 600 which provides reluctance against relative axial motion of the carrier 300 relative to the housing 110/120 where the reluctance is greater at higher speeds than at lower speeds.

While the carrier spring device in the shown embodiment is provided as a compression spring 680, other spring devices may alternatively be used such as a torsion spring that is coupled to rotatable member 640 and that, when the rotatable member has been rotated away from its initial position, serves to move the rotatable member 640 back towards its initial position and hence move carrier 300 back to its initial axial position. In still alternative embodiments, the spring action provided by the carrier spring device may be obtained by other means such as an elastic foam member, a pneumatic or hydraulic spring device, a device utilizing magnetic force or the like.

In each of FIGS. 3a-3e, the device 100 is shown having the distal face of the needle shield 150 abutting a surface “S” representing an injection site such as the skin surface of a patient. The FIGS. 3a through 3e show the device 100 in different states during operation of the device. FIG. 3a depicts the device 100 in a state corresponding to the state shown in FIG. 1, i.e. where the device is in its initial state after the protective cap has been removed but prior to administration. However, compared to FIG. 1, FIG. 3a includes further reference to details that mainly deals with sequential control of movement of various parts of the device.

The injection device 100 includes a carrier detent mechanism 151, 152, 115, 153, 353 which ensures that movement of the needle shield 150 is accompanied by movement of the carrier 300 but only when the needle shield 150 is located at its distal position and in positions ranging between the distal position and an intermediary position located between the distal position and the proximal position (all these positions referring to the position of the needle shield 150 relative to the housing 10/120).

The injection device 100 also includes a release trigger 311, 312, 313, 403 that serves to maintain the piston driver 400 arrested relative to the carrier 300 when the device in a pre-triggering state and when the needle shield 150 is located between the distal position and a trigger position located proximal to the intermediary position.

Having regard to the release trigger, a trigger control member 311 formed as a longitudinally extending flexible arm is formed integral with the carrier 300. Trigger control member 311 extends in the proximal direction away from a bridging portion of the remainder of the carrier and defines a proximal free end being adapted to be flexed in the radial direction. Trigger control member 311 has an inherent tendency to move the free end of the trigger control member radially outwards. The free end of trigger control member 311 includes a radially inwards protruding surface 313 and a radially outwards facing surface 312. The radially inwards protruding surface 313 of the free end of the trigger control member 311 is in FIG. 3a shown to engage an enlarged portion 403 of the piston driver 400. When the inwards protruding surface 313 engages the enlarged portion 403 the piston driver 400 is maintained in the initial position relative to carrier 300 against the force of the first compression spring 500.

The radially inwards facing wall surface of the needle shield 150 includes a recessed region formed as an opening 155 in the wall surface which is adapted to cooperate with the radially outwards facing surface 312 of the free end of the trigger control member 311.

When the injection device 100 is in the state prior to triggering, such as shown in FIG. 3a through 3c, an inner wall surface of the needle shield 150 located proximally to said opening 155 engages the radially outwards facing surface 312 of the trigger control member 311 and acts to maintain the free end of trigger control member 311 pushed radially inwards so that the radially inwards protruding surface 313 is maintained in engagement with the enlarged portion 403 of piston driver 400. Hence, as long as the needle shield 150 is located relative to the carrier 300 so that the surface 312 of the trigger control member 311 is located proximally to opening 155, piston driver 400 cannot move to expel the medicament from the medicament reservoir 200.

However, upon manipulating the needle shield 150 to trigger the expelling operation, when the opening 155 of the needle shield has been axially aligned with the surface 312 of the trigger control member 311, the free end of trigger control member 311 moves radially outwards. This makes the radially inwards protruding surface 313 move out of engagement with the enlarged portion 403 of piston driver 400 thereby releasing the piston driver 400 enabling the first compression spring 500 to drive the piston driver in the distal direction for expelling a dose of medicament from medicament reservoir 200.

In the shown embodiment, having regard to the above mentioned carrier detent mechanism 151, 152, 115, 153, 353, a carrier control member 151 formed as a longitudinally extending flexible arm is formed integral with the needle shield 150. Carrier control member 151 extends in the proximal direction away from a bridging portion of the remainder of the needle shield and defines a proximal free end being adapted to be flexed in the radial direction.

When the injection device 100 is in the state shown in FIG. 3a, carrier control member 151 has an inherent tendency to move the free end of the carrier control member 151 radially outwards. The free end of carrier control member 151 includes a radially inwards protruding surface 153 and a radially outwards facing surface 152. In FIG. 3a the radially inwards protruding surface 153 is shown to engage the recessed region 353 of carrier 300. When the inwards protruding surface 153 engages the recessed region 353, carrier 300 is forced to move axially together with the needle shield 150.

When the injection device 100 is in the initial state, an inner wall surface of top housing 120 engages the radially outwards facing surface 152 acting to maintain the free end of the carrier control member 151 pushed radially inwards into engagement with the recessed region 353 of the carrier 300. Hence, as long as the needle shield 150 is located relative to the housing 110/120 between the distal position and the intermediary position movement of the needle shield 150 is accompanied by movement of the carrier 300 and thereby also the medicament reservoir 200 as well as the injection needle 250.

When the needle shield 150 is positioned at the intermediary position and positions proximal to the intermediary position, a recessed region 115 formed on an inner surface of top housing 120 allows the free end of carrier control member 151 to move radially outwards so that the inwards protruding surface 153 is disengaged from the recessed region 353 of carrier 300. When this occurs the needle shield 150 is free to move axially relative to the carrier 300.

FIG. 3b shows the device 100 where the needle shield 150 has been moved proximally to a position slightly distal to its intermediary position relative to the housing 110/120. Hence, the carrier control member 151 is still pressed radially inwards by means of the inner wall surface of top housing 120.

As the needle shield 150 has moved from the distal position to the intermediary position and as the carrier 300 and the carrier end member 650 have been travelling together with the needle shield 150, the second compression spring 680 has been compressed relative to its initial state. When the needle shield 150 is moved from the distal position to the intermediary position, the carrier 300, medicament reservoir 200, injection needle 250, piston driver 400 and first compressible spring 500 all move with the needle shield 150 relative the housing 110/120. Thus the weight of components which is rigidly connected to the housing is comparatively low. Hence, in case the injection device is dropped on a hard surface resulting in a proximally directed impact on the needle shield 150 the risk of such impact resulting in the device being unintentionally triggered is low.

The movement of the carrier end member 650 relative to the housing 110/120 is accompanied by rotation by the rotatable member 640. This will additionally dampen a potential impact so that the forces arising from the impact will to a lesser degree be transferred to act for triggering the injection device 100. This is particularly true for impacts involving high velocity movements. However, for a deliberate triggering movement such as when a user carries out an administration where the velocity of movement is low, the damping effect of the impact damper 600 is low. Should the force that presses the needle shield 150 towards the intermediary position cease, the energy accumulated in compressible spring 680 will act to return needle shield 150 and carrier 300 into their initial position corresponding to the state shown in FIG. 3a.

FIG. 3c shows the injection device 100 where the needle shield 150 has been moved further proximally past the intermediary position to a position slightly distal to the trigger position. As mentioned above, when the needle shield has entered into the intermediary position, the carrier control member 151 is disengaged from the carrier 300. Hence, for all needle shield positions from the intermediary position and towards proximal position, the needle shield 150 is free to move axially relative to the carrier 300. Due the disengagement occurring at the intermediary position, the carrier 300 is not forced to move further proximally and in fact, in the shown embodiment, will be prevented from doing so by the carrier end member 650 abutting a surface of the top housing 120. The relative movement of the needle shield 150 relative to carrier 300 is accompanied by the needle shield spring 160 being increasingly compressed. Ultimately, for some embodiments, the compression of the needle shield spring 160 will at some point prevent the carrier 300 from being moved further distally relative to the needle shield 150.

In FIG. 3c the movement of the needle shield 150 relative to the carrier 300 has brought the front part of the injection needle 250 to protrude through the aperture 159 of the needle shield and protrude partly into the surface “S”. During manipulation of the needle shield 150 relative to the housing 110/120 the injection needle 250 is brought to gradually protrude through the aperture 159 as the needle shield 150 is moved. Hence, at least for the initial part of the needle insertion process, as the needle insertion into the skin is performed manually, the user of the injection device 100 is given full control of the needle insertion process. However, in FIG. 3c, the injection device 100 has not yet been triggered for the dose expelling procedure, due to the trigger control member 311 still being pressed radially inwards.

In FIG. 3d, the needle shield 150 has been moved further proximally slightly past the trigger position, the injection needle 250 protrudes further into the skin and the needle shield spring 160 is fully compressed. In the trigger position, the trigger control member 311 aligns axially with the recessed region 155 of the needle shield sot that the free end of trigger control member 311 is moved radially outwards. As a result the radially inwards protruding surface 313 is disengaged with the enlarged portion 403 of piston driver 400. Hence, as shown in FIG. 3d, piston driver 400 is released and has started to move in the distal direction by means of the force exerted by first compression spring 500 to push the piston 220 to expel the contents of the medicament reservoir 200.

FIG. 3d shows the injection device 100 at the end of the expelling procedure where a mechanical stop has limited further axial movement of piston driver 400. The stop feature is not shown but may in some embodiments be provided as a mechanical stop formed integral with the piston driver 400 that engages the proximal end section of the medicament reservoir 200. In other embodiments, the piston end position may be defined by the piston hitting an end stop included in the medicament reservoir such as the reduced neck portion 215 (cf. FIG. 1).

At the end of the expelling movement, the injection device 100 may be removed from the injection site whereafter the needle shield 150 will move distally relative to the housing 110/120 by means release of the energy accumulated in the needle shield spring 160 and optionally also in the second compression spring 680. The distal movement of the needle shield 150 may be accompanied by a lock feature (not shown) to provide a permanent locking of the needle shield 150 in a distal position so that the injection needle 250 is made permanently inaccessible subsequently to the medicament administration.

FIG. 4 shows an injection device 100 according to a second embodiment, which basically functions similarly to the previously described first embodiment but where the medicament reservoir with the integrally formed injection needle 200/250 discussed in connection with the first embodiment has been replaced by a medicament reservoir in the form of a cartridge 1200 and a separate injection needle 1250. In FIG. 4 the parts that correspond to similar parts in the first embodiment shares the same reference numbers and the parts performs correspondingly except for the following.

In the second embodiment, the narrowed neck section 1250 of the medicament cartridge is closed off by a pierceable septum 1230 which prior to administration maintains the contents of the cartridge 1200 in a sealed condition. The injection needle 1250 includes a hub section which holds a front needle 1251 adapted to pierce the skin of a patient and a rear needle 1252 adapted to pierce the pierceable septum 1230 of the cartridge. Typically the front needle 1251 and the rear needle 1252 are integrally formed as a single component. The hub section of the injection needle 1250 is connected to the carrier 300 by means of a suitable connection that may be established at the time of manufacturing the device or may be established immediately prior to administration by the user of the device.

In the second embodiment, the cartridge 1200 is adapted to axially slide from a first proximal position relative to the carrier 300 and into a second distal position relative to the carrier. This sliding movement is provided for bringing the cartridge 1200 into fluid communication with the injection needle 1250 but only upon triggering of the device.

FIG. 4 shows the device according to the second embodiment in the initial state where the device is ready to be positioned relative to an injection site and thus in a state corresponding to the state of the first embodiment shown in FIG. 3a. In the initial state, the cartridge 1200 is positioned in the carrier 300 so that the cartridge septum 1230 is adequately separated relative to the rear needle 1252 so as to maintain the cartridge sealed prior to triggering of the device 100.

In some embodiments, the piston 1220 of the cartridge may be attached to the piston driver 400 such as by mechanically engaging cooperating features to ensure that the cartridge 1200 is axially separated relative to the rear needle 1252. Alternatively, the cartridge 1250 may be retained axially relative to the carrier 300 by means of friction elements formed internally in carrier 300 positioned to prevent the cartridge 1200 from moving distally from its proximal position relative to carrier 300 unless the full force from the first compression spring 500 is released to act on the piston driver 400, i.e. upon triggering of the injection device 100.

The operational procedure of the second embodiment fully corresponds to the first embodiment until the point of triggering, i.e. in correspondence with the procedural steps shown in FIG. 3a-3d. However, in the second embodiment, when the piston driver 400 is released, the energy accumulated in the first compression spring 500 initially drives forward the piston driver 400 to move the cartridge 1200 axially from its proximal position to the distal position relative to the carrier 300 to thereby make the rear needle 1252 penetrate the pierceable septum 1230. This movement establishes fluid communication between the cartridge and the injection needle. When the cartridge 1200 enters into its distal position the cartridge movement is halted by means of a proximal surface of the carrier 300. Subsequently, the first compression spring 500 pushes the piston driver 400 so that the piston 1220 is driven in the distal direction relative to cartridge body 1210 and so that medicament accommodated in the cartridge 1200 is expelled through the injection needle 1250. Hereafter, the injection device 100 may be removed from the injection site, and the needle shield may enter into a position that maintains the front needle 1251 inaccessible.

FIG. 5 shows an injection device according to a third embodiment which fully corresponds to the first embodiment except that the damper 600, i.e. parts 640 and the threaded connection between elements 347 and 647 has been omitted. As regards safety measures against unintentional firing of the injection device, such as the potential risk that the injection device is dropped on a hard surface, the third embodiment fully relies on the suspension configuration between the carrier 300 and the housing 110/120 where the second compression spring 680 is adapted to take up the energy of an impact. As noted above the

FIG. 6 through 8 show embodiments of an injection device incorporating different variants of an impact damper but where the respective injection devices basically function similarly to the previously described first embodiment.

FIG. 6 shows an injection device 100 according to a fourth embodiment where the rotational damper 600 of the first embodiment has been replaced by an air damper 1600. Air damper 1600 is formed between the carrier 300 and the top housing 120 to provide a damping function for rapid relative movements between carrier 300 and housing 110/120. Air damper 1600 is defined by a variable chamber 1630 having wall parts provided by top housing 120 as well as wall parts provided by a component 1620 connected to or integrated with carrier 300. In the shown embodiment, the component 1620 is provided with a seal 1622 to provide an air tight seal at the interface between the walls of the two components. An air leak valve 122 is provided it top housing 120 to provide a controlled seepage of air from the variable chamber 1630.

Should an unintentional impact on needle shield 150 occur, then air damper 1600 is able to limit part of the impact forces to be transferred into movement of the needle shield and thus limit the extent of movement of the needle shield. However, when the injection device 100 is applied to an injection site such that the needle shield is slowly pressed in the proximal direction relative to housing 110/120, the air damper allows the variable chamber 1630 to be reduced at a suitable rate due to the well-defined air resistance of air leak valve 122. Hence, when the needle shield 150 is pressed slowly in the proximal direction, the needle shield 150 is allowed to progress as far as to the intermediary position relative to the housing 110/120. Hereafter the needle shield can move further in the proximal direction for bringing the device into the triggered state. This latter movement is unrestricted by the air damper 1600 and the carrier spring device (compression spring 680) due to the needle shield 150 has become disengaged relative to the carrier 300.

FIG. 7 shows a fifth embodiment with a further variant 2600 of a damper mechanism. In the fifth embodiment, a foam element 2630 is arranged between the top housing 120 and a component 2620 connected to or integrated with carrier 300. The foam element 2630 is compressed when the carrier 300 is forced to move proximally relative to the housing 110/120. The type of foam material of foam element 2630 is selected so that foam element is able to provide as an impact absorber. Hence, foam element 2630 mainly dissipates energy from forces arising at sudden impacts on the needle shield 150. In some variants of the injection device the foam element 2630 additionally acts as the carrier spring device. In that case the second compression spring 680 may be omitted. In particular embodiments, the type of foam is selected as a foam type which provides increased resistance against compression at large velocities compared to the resistance against compression at low velocities.

Finally, FIG. 8 shows a sixth embodiment of an injection device 100 wherein a damper 3600 is provided as a grease damper. Top housing 120 is formed at its proximal end as a cylindrical hollow with an internal diameter sized to fit a slideable cylinder element 3620 that is connected to or formed integral with carrier 300. The interfacing walls of the cylindrical hollow and the cylinder element 3620 is provided with grease to provide resistance when the carrier is moved relative to the housing. Similar with the other embodiments described herein, the grease damper 3600 may be designed to provide an increased resistance against relative movement between carrier 300 and housing 110/120 at large velocities compared to the resistance occurring at low velocities.

In each of the third through sixth embodiments, the configuration of the medicament reservoir 200 and the injection needle 1250 may be replaced by a configuration wherein the cartridge 1200 is separate from the injection needle 1250 such as described in connection with the second embodiment.

Also the function of the needle shield lock as briefly discussed in connection with the first embodiment may be incorporated into the devices according to the second through sixth embodiments.

In other embodiments, in accordance with the invention, all of the above injection devices may in alternative forms be configured for multiple separate injections whereby the needle shield is adapted multiple times to be moved back and forth relative to the housing from a distal position to a proximal position where the carrier moves with the needle shield from the distal position to an intermediary position and where the needle shield is decoupled from the carrier to allow the needle shield to move further proximally independent from the needle carrier. During the latter movement the injection needle is brought to protrude from the needle shield whereafter the injection device is triggered for dose expelling by moving the needle shield even further proximally.

In still other embodiments according to the invention, the size of the dose of the single dose, or, in case the injection device is configured for multiple injections, the size of individual doses may be adjustable by the user by means of a dose setting feature whereby the user may adjust the size of a dose that is subsequently expelled.

Finally, all the embodiments described herein may include sealing functions for the injection needle so that prior to administration the tip point of the injection needle may be covered by a needle sheath that maintains the injection needle in a sterile condition. The needle sheath of the front needle may be of a kind which is penetrated when the injection needle is moved relative to the needle shield. In alternative embodiments the needle sheath is of a kind where the needle sheath is removed from the injection needle prior to administration. In embodiments where the injection needle comprises a rear needle, such needle may also include a penetrable needle sheath that prior to administration maintains the rear needle in a sterile state and wherein the needle sheath is configured to be penetrated when the rear needle and the cartridge moves relative to each other when fluid communication is to be established.

1. An injection device for administering a dose of a medicament from a held medicament reservoir, the injection device comprising:
a housing defining distal and proximal ends,
an injection needle arranged at the distal end of the housing and adapted for fluid communication with a held medicament reservoir,
a needle shield disposed at the distal end of the housing, the needle shield being movable proximally relative to the housing upon exertion of an externally applied force to gradually expose the injection needle as the needle shield moves proximally relative to the housing, and
an actuator configured to act on the medicament reservoir to expel a dose of medicament therefrom,
wherein the injection device further comprises:
a carrier that is movable relative to the housing from a distal position to a proximal position, the carrier holding the injection needle and the medicament reservoir,
a carrier spring device arranged between the housing and the carrier, the carrier spring device being configured to force the carrier in the distal direction, and
a detent mechanism arranged between the carrier and the needle shield, wherein the detent mechanism is configured to:
couple axial movement of the needle shield with axial movement of the carrier as the needle shield moves proximally relative to the housing from a distal position to an intermediary position so that the injection needle is maintained in a shielded state, and
allow relative axial movement between the needle shield and the carrier as the needle shield moves relative to the housing from the intermediary position and into a proximal position so that the injection needle is gradually exposed as the needle shield (M) moves relative to housing between the intermediary position and the proximal position.

2. The injection device as defined in claim 1, wherein the detent mechanism is configured to prevent relative axial movement between the carrier and the needle shield when the needle shield is positioned between the distal position and the intermediary position.

3. The injection device as defined in claim 1, wherein the detent mechanism is coupled to the needle shield and the carrier to lock the carrier relative to the needle shield and wherein a carrier control member is arranged to cooperate with the housing so that relative movement between the needle shield and the housing acts to release said lock upon the needle shield entering into the intermediary position.

4. The injection device as defined in claim 1, wherein the needle shield is positionable relative to the housing into a trigger position, the trigger position being located proximally to said intermediary position, wherein the actuator is maintained in an inactive state when the needle shield is positioned distally relative to the trigger position, and wherein the actuator is released from said inactive state when the needle shield is moved proximally into the trigger position.

5. The injection device (100) as defined in claim 1, wherein the needle shield is so configured that movement of the needle shield acts to release the actuator in a state where relative movement between the needle shield and the carrier has shifted the injection needle into the state where the injection needle is exposed.

6. The injection device as defined in claim 1, wherein the actuator comprises a drive spring configured for forwarding load of the drive spring to the piston of the medicament reservoir to expel a dose of a drug medicament from the medicament reservoir upon release of the actuator.

7. The injection device as defined in claim 1, wherein a needle shield spring device is coupled to the needle shield and configured to bias the needle shield towards its distal position.

8. The injection device as defined in claim 1, wherein an impact damper is arranged between the housing and the carrier, the impact damper being configured to dampen motion of the carrier relative to the housing.

9. The injection device as defined in claim 8, wherein the impact damper is configured as a damper mechanism incorporating one or more damper mechanisms selected from the group consisting of a hydraulic damper, a pneumatic damper, a grease damper, a rotational motion damper and a foam damper.

10. The injection device as defined in claim 1, wherein the medicament reservoir defines an elongated reservoir body, wherein an injection needle is mounted on said reservoir body at a distal end thereof and wherein a slideable piston is arranged within the reservoir body to be driveable in the distal direction to expel medicament from the medicament reservoir.

11. The injection device as defined in claim 1, wherein the medicament reservoir, in a situation of use, is fixedly mounted relative to the carrier.

12. The injection device as defined in claim 1, wherein the medicament reservoir defines a medicament cartridge having an elongated cartridge body, wherein a cartridge septum seals the distal end of cartridge body, the cartridge septum configured for being pierced by the injection needle for establishing fluid communication with medicament contained in the medicament cartridge and wherein a slideable piston is arranged within the cartridge body to be driveable in the distal direction to expel medicament from the medicament cartridge.

13. The injection device as defined in claim 12, wherein the injection needle defines a front needle for penetrating the skin of a subject user and a rear needle for piercing the cartridge septum, the front needle and rear needle being configured for fluid communication, and wherein the injection needle is mounted fixedly relative to the carrier to be moveable together with the carrier.

14. The injection device as defined in claim 13, wherein the medicament cartridge is arranged within the carrier so that, prior to triggering of the injection device, the rear needle is separated axially from the cartridge septum.

15. The injection device as defined in claim 14, wherein the medicament cartridge is arranged slideable within the carrier so that when the actuator acts on the medicament cartridge, the medicament cartridge initially slides relative to the carrier so that the cartridge septum is pierced by the rear needle and subsequently the piston is driven by the actuator in the distal direction relative to the cartridge body 1210 to expel medicament from the medicament cartridge.
